(19) United States
(12) Patent Application Publication
(10) Pub. No.: US 2019/0133926 A1
(43) Pub. Date: May 9, 2019

(54) Burn Pill Pharmaceutical Smoking Cylinder

(71) Applicant: Tremaine Jackson, West Covina, CA (US)

(72) Inventor: Tremaine Jackson, West Covina, CA (US)

(21) Appl. No.: 15/805,279

(22) Filed: Nov. 7, 2017

(51) Int. Cl. A61K 9/00; A61K 9/20; A61M 15/06; A61M 11/04

(52) U.S. Cl. CPC A61K 9/007; A61K 9/2077; A61K 9/2013; A61M 11/041; A61K 9/2068; A61M 15/06; A61K 9/2009

(57) ABSTRACT

A medicated pill having a composition comprising of the active pharmaceutical agent (API) in a concentration in the range of 5% to 40% by weight and excipients in a concentration in the range of 20% to 80% by weight, based on the total weight of the pharmaceutical composition.

### FIELD OF THE INVENTION

The present invention talks of a unique medicated pill that presents the feature of getting burnt as that of a cigarette with the medicated vapor getting inhaled through lungs to be absorbed in the blood-stream. The burnt pill utilizes Active Pharmaceutical Ingredient (API) along with excipients, burnt within a smoking cylinder which is selected from various groups e.g., Antibiotics, antipyretics, vitamins, herbs etc.

### BACKGROUND OF THE INVENTION

Administering of drugs can be through a wide variety of routes. Primary ones are the enteral routes (e.g., sublingual, oral, and rectal), parenteral injections (e.g., subcutaneous, intramuscular, and intravenous), inhalation, topical, transdermal and intranasal.

Thus, for administering a drug, to be able to produce the intended clinical effects, it must first be able to reach its target site of action within the body at an effective concentration. For drugs meant for producing an effect inside the body, be it widespread (e.g., systemic antibiotics) or specific (e.g., anti-thyroid agents), those must be administered in ways to enable systemic circulation and appropriate transportation to the damaged site (s) where the deranged body function needs to be rectified.

Of-late, quite a number of new technologies in virtually every area of drug delivery, including transmucosal, skin delivery, and inhalation, in addition to oral drug formulations have been introduced. Amongst these, inhalation techniques ensure some specific types of drugs that can be vaporized allow rapid drug delivery through deep lung inhalation. In such a process, the drug is quickly absorbed through the lungs into the bloodstream, providing a speed of onset of activity that is comparable to that of intravenous administration.

For smokers, deploying such inhalation techniques can be an easy way of administering drugs utilizing their usual habit synced with their requirement of a fast-acting drug. The present invention describes one such drug that is in the form of a pill which when burnt can be administered through smoking.

### PRIOR ART

To establish the uniqueness of the present innovation, a discrete survey of similar patents revealed the following:

In the US patent titled, “Therapeutic smoking device” (U.S. Pat. No. 8,490,629 B1), talks of a smoking device intended for therapeutic uses, such as medical marijuana or herbal remedies, providing for cooling of smoke due to latency in multiple chambers, filtration of condensed tars from cooled smoke, and decreased respiratory strain for the user provided by a annular carburetor valve, which propels the cooled and filtered smoke into the user's airstream by means of pressure equalization is discussed. In a preferred embodiment, the device is constructed so as to be extremely durable, shielding the glass smoking bowl from impact and shock, and is manufactured of non-toxic materials appropriate to a device for therapeutic use. A venturi effect chasing of entrapped smoke projects the smoke quickly into the lungs. The taste of different herbs such as tobacco, cannabis indica, cannabis sativa and different strains of each is accentuated.

In another patent titled, “Ayurvedic, herbal smoking composition” (US 2005/0279373 A1) discussion on a smoking material that comprises a dried preparation of Fenugreek leaves mixed with other powdered spices and honey are taken up. This smoking material can be used alone or can be combined into mixtures with smoking tobacco, if desired. The smoking composition, which is aromatic is free of nicotine and contains health-promoting herbs.

In the next invention titled, “Medicinal delivery system, and related methods” (U.S. Pat. No. 8,642,016 B2), a method is provided of making a medicinal delivery system which satiates a craving in an individual when the medicinal delivery system is administered orally to the individual. A coating composition is applied on a saliva-soluble powder to establish a coated powder, the coating composition featuring an at least partially solubilised craving satiation medicinal compound. The coated powder is combined with an edible carrier base to establish a medicinal delivery system that rapidly releases medicine and buffer preferably followed by slower, sustained release.

However, in all these invention, the usage of a burning pill to generate medicinal vapour has not been dealt with.

### SUMMARY OF THE INVENTION

An object of the present invention is to provide a medicated pill whose active ingredients have the physical property to sublime (from solid to gas) or evaporate (from liquid to gas) during the smoking process

An object of the invention is to provide a medicated burn pill that when used entails the sublimation of substances with medicinal value into vapors, and the administration of these substances into the bloodstream through inhalation via the lungs. These active ingredients are employed to treat various minor ailments, such as muscle pain, headache, migraine, infection and/or for relaxation purposes without limitation.

Another object of the present invention is to provide a medicated pill where the active pharmaceutical ingredient is chemically stable under smoking conditions. The API must not decompose or degrade at the temperature range used during combustion. This decomposition or degradation of active ingredients might result in loss of activity and/or generation of harmful degradation products. For example, if the heating range of the vaporizer is 150° C. to 200° C., then the temperature at which the decomposition of the active ingredient starts must be significantly higher. The API should transform to vapors—by heating within the temperature range—that are inhaled through a filter.

Still another object of the present invention is to provide a medicated pill, wherein the additives in the pill do not produce tars, thereby rendering the pill safe for inhalation by the user.

Another object of the present invention is to provide a drug which can be administered through inhalation, which ensures almost instantaneous absorption and very rapid onset.

A still further object of the invention is to make available a drug, which might produce localized effect to lungs with minimal systemic side effect.

Another object of the present invention is the fast release of API and administration of APIs through smoking, wherein, the time elapsed until the API reaches active blood level should range between 60 s and 300 s.

Another object of the present invention is to provide an alternative and more comfortable means to deliver medications to the user. This can be illustrated in cancer patients, who usually suffer from disturbances in their gastrointestinal system due to chemotherapy, benefits from administration of drugs via inhalation. In addition, administering drugs via inhalation may alter the metabolism of the active ingredients due to the avoidance of the GIT and liver. This may lead to higher blood levels after administering doses smaller than those given orally.

Another object of the present invention is to provide a smoking cylinder which accommodates the medicated pill of the present invention and releases the medicated smoke in a controlled manner in the body of the user.

A still further object of the invention is to provide a smoking cylinder accommodating the medicated pill of the present invention, which can be designed both as a perishable as well as a non perishable form.

### DESCRIPTION OF THE DRAWINGS

FIGS. 1a and 1b illustrates the smoking cylinder of the present invention with the burn pill inserted in front of the cylinder.

FIG. 2 illustrates the cotton filter of the smoking cylinder of the present invention.

FIGS. 3a and 3b illustrating a larger Burn Pill dose inserted into the opening of a smaller cylinder for smoking for admitting larger amounts of Burn pill of the present invention to an acute patient.

### DETAILED DESCRIPTION OF THE PREFERRED EMBODIMENTS

The detailed description of preferred embodiments are examined from three perspective

Design and composition of the smoking cylinder;

The design of the pill;

Composition of the medicated pill.

### Design of the Smoking Cylinder

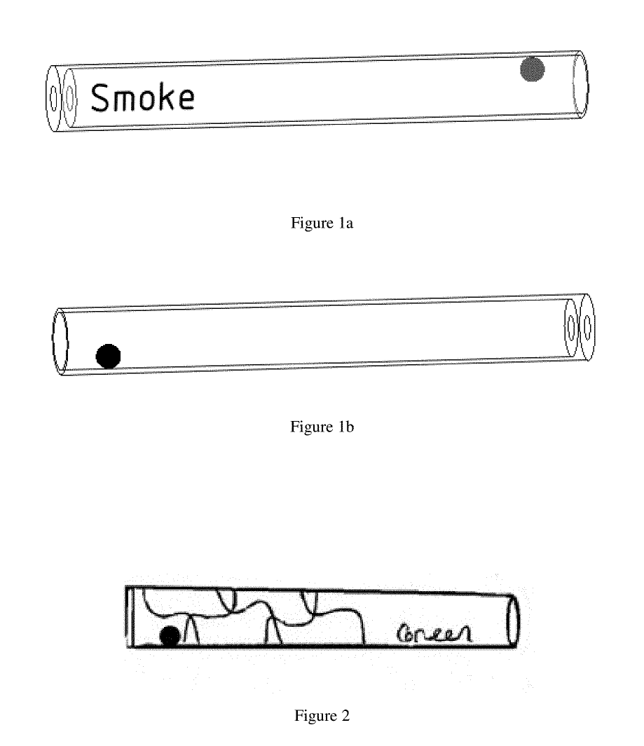

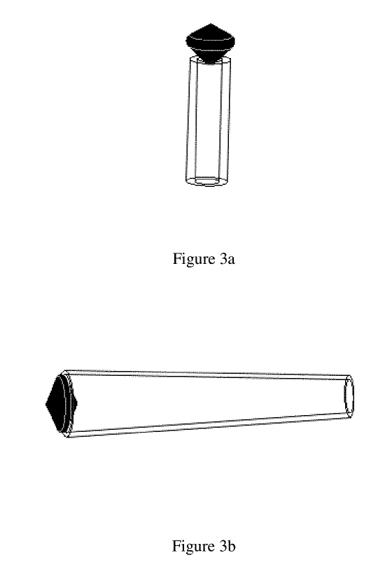

Referring now to FIGS. 1-3, the smoking cylinder of the present invention will be described in detail, which has a length between 50 and 250 mm depending upon the type of dosage of the pill and the condition of the user. The inner diameter of the said smoking cylinder is between 5 and 20 mm.

The tip of the smoking cylinder is provided with a piece of cotton (or any other similar material) saturated with a mixture of inorganic salts and/or polymers for increasing the absorption/adsorption of harmful tars and aldehydes.

These polymers are selected from group including, but not limiting to nitroxide radicals-containing polymer (NRP), comprising of poly(4-methylstyrene), N,N′-bis (3-triethoxysilylpropyl)thiocarbamide organo-silicon, and the inorganic salts are selected from group including alkali ferrates and/or herbal-based materials.

The smoking cylinder of the present invention releases the drug in a controlled manner through a specific design of the internal structure. It comprises a chamber supplied by a heating element for burning the pill. The heating chamber opens on another chamber that contains the filter, and ends with an orifice delivering the vapor to be inhaled to the user.

In another embodiment of the present invention, the smoking cylinder is perishable, which, after one use can be discarded. In such embodiments, the material used is usually comprises of paper, made of hemp.

In another embodiment, the smoking cylinder is made of metal or glass and thus is reusable.

For reusable smoking cylinder, the materials of choice are metals or glass. In embodiments where the smoking cylinder is made of metal, for reducing the transfer of heat to the user, the wall of the cylinder is usually double layered.

When the material used is glass, it is usually selected from the group which has high resistance to thermal shock brought forth through usage of borosilicate glass and quartz glass.

In an embodiment, the above blended burn-pill compound comprises of dyed green tobacco and hemp blend devoid of any T.H.C. hallucinogen properties or tars, where the compound is softened and enclosed in paper or leaves, which are coated from inside with fish oil, sea weed and or alkaline water depending on the variation of the compound in the cylinder roll without limitation. Usage of these coatings ensures advantageous health benefits on account of their medicinal and nutritional values. As will be known to those skilled in the art, these coated ingredients have some specific advantage which are enumerated as under:

Fish oils: Fish oil is known to be beneficial for brain and retina development. It is also protective against cardiovascular diseases, inflammatory conditions and mental disorders. Also it has been reported that its selenium content is responsible for the antagonistic effect it has on mercury toxicity and the conciliating effect on prostate cancer.

Seaweeds: are rich in volatile antioxidants and iodine. The vapors are considered beneficial in the treatment of iodine deficiency and for protection against cancer and Alzheimer disease. Seaweeds have antiviral, anti-coagulant and anti-oxidant properties and are protective against cardiovascular and ocular inflammatory diseases. They contain iodine which regulates thyroid levels in the body. They are also rich sources of polysaccharides which inhibit fat digestion thereby reducing obesity. The polysaccharides help in maintaining healthy digestive system and also imparts anti-diabetic effects.

Alkaline water: has a higher pH level than regular drinking water which usually ranges between pH of 8 to 9 and provides useful levels of many essential minerals like Calcium (Ca2+), Magnesium (Mg2+), Iron (Fe3+), Sodium (Na+), Zinc (Zn2+), Copper (Cu+ or Cu2+), Potassium (K+), Molybdenum (Mo+), Manganese (Mn2+). The vapors are considered to be beneficial in the treatment of blood pressure, diabetes, and cholesterol. Besides, Alkaline water has anti-aging properties; colon-cleansing properties, immune system support hydration, skin health, and other detoxifying properties apart from aiding in weight loss and having cancer resistive properties.

In some embodiments instead of the above-mentioned ingredients discussed, the blended compounds contain fruits, vegetables, and or wheat or lemon grass extracts, or caffeine stimulants for smoking. Caffeine is commonly used in combination with pain killers since it acts as a stimulant, improving mental alertness whereas fruits and vegetable serve as flavoring agents.

As will be known to those skilled in the art, wheat and lemon grass which are also used as ingredients of the composition of the present invention have certain beneficial properties which are enumerated as under:

Wheatgrass: provides local therapeutic effect to the respiratory tract to relieve conditions such as cough, common cold, sore throat and bronchitis. It further increases hemoglobin production in the body and improves diabetic, hypertensive and hyperlipidemia conditions. It has wound healing properties and prevents bacterial infections.

Lemongrass: contains high levels of terpens such as geraniol, myrcene, nerol and citral. Its vapors possess significant anti-inflammatory, antimicrobial and immunity-boosting characteristics. In the present invention, the dose used will ranges between 5 to 50% of the final pill weight.

### Design of the Pill

The medicated pill of the present invention is designed with a shape and geometry to fit into the cylindrical tube as described in FIGS. 1-3. The shape and size usually depends on the final use of the pill. Depending upon the purpose of the pill, it may be either rigid solid or flexible resin like solid.

In an embodiment of the invention, the pill is shaped cylindrically wherein the diameter ranges between 3 mm and 8 mm and the length ranges between 5 and 15 mm.

In another embodiment of the present invention, the pill is shaped like a disc, wherein, the diameter ranges between 8 and 15 mm and the thickness ranges between 3 and 10 mm

### Composition of Pill

The medication of the present invention has a pharmaceutical composition for administration by inhalation, comprising of an active agent and an optional pharmaceutically acceptable diluents and/or excipients.

As will be known to those skilled in the art, active agent or Active Pharmaceutical Ingredient (API) comprises of a typical substance or mixture of substances primarily intended to be used in the manufacture of a drug product. Such substances are intended to furnish pharmacological activity or other direct effect in the diagnosis, cure, mitigation, treatment or prevention of diseases or to affect the structure and function of the body.

The active pharmaceutical ingredient (API) of the present invention is selected from either of the following groups e.g., Antibiotics, antipyretics, vitamins, herbs etc.

Antibiotics are antibacterial drugs produced from microorganisms preventing or treating infections. There are different drugs belonging to this class of drugs and they vary in chemical nature and mechanism of action.

The antibiotics of the present invention is selected from the group comprising β-lactams, tetracyclines, macrolides, aminoglycosides, anti-metabolites, nucleic acid analogs, antifungals and antivirals.

Antipyretics are drugs for reducing fever by inducing a reduction of body temperature. These are selected from the group of Non-steroidal anti-inflammatory drugs (NSAID). Also included are acetaminophen and aspirin.

Vitamins are considered as dietary supplements, providing nutritional benefits and maintaining chemical balance of the body.

Herbs are natural plant products having pharmacological actions. Some herbs have nutritional benefits and some others act as flavoring agents.

Besides, Active Pharmaceutical Ingredient (API) the optional excipients which are basically pharmaceutical additives having inactive ingredients are used to make up the burn-pill. The stability as well as release characteristics of the drug in formulation depends mainly on the quality and quantity of excipients and therefore, the selection of excipients is a crucial step towards developing a stable and successful dosage form. Excipients are classified as organic as well as inorganic excipients and also as vaporizing vehicle. The present invention in their various embodiments uses both the varieties of excipients as well as a vaporizer in their medical formulation.

These are selected from the group comprising cellulose and its derivatives, starch and its derivatives, waxes, herbs, etc. Some of the features of the excipients used, as will be known to those skilled in the art are enumerated below:

Starch: as an excipient is used for nasal, oral, periodontal, and other site-specific delivery systems. It is known in the art that chemically, starches are polysaccharides, composed of a number of monosaccharides or sugar (glucose) molecules linked together with α-d-(1-4) and/or α-d-(1-6) linkages. Starch consists of 2 main structural components, the amylose, which is essentially a linear polymer in which glucose residues are α-d-(1-4) linked typically constituting 15-20% of starch, and amylopectin, which is a larger branched molecule with α-d-(1-4) and α-d-(1-6) linkages and a major component of starch.

Wax: is chemically defined as an ester of a monohydric long chain fatty alcohol and a long chain fatty acid. They usually contain a wide variety of materials including glycerides, fatty alcohols and fatty acids and their esters.

Cellulose: on account of their compatibility with the most of other excipients, is of pharmacologically inert nature and indigestable by human gastrointestinal enzymes. Further, these polymers do not have any irritancy potential on stomach and esophagus protective mucosa.

Apart from these, herbal excipients also have various advantages as:

Biodegradable: since herbal excipients are naturally occurring polymers produced by living organisms, they have no adverse effects on the environment or human beings.

Biocompatible and non-toxic—Chemically, nearly all of these plant materials/herbal excipients are carbohydrates in nature and composed of repeating monosaccharide units. Hence they are non-toxic.

Economic—These are also cheaper and their production cost is less than synthetic material.

Safe and devoid of side effects—These are from a natural source and hence, safe and without side effects.

The inactive ingredients includes, but is not limiting to calcium or sodium salts, Magnesium stearate, talc, lactose, and titanium dioxide, etc. Other inactive excipients are employed to improve texture, consistency and the efficacy of the final product. The inactive ingredients weighs not less than 50% of the final pill weight.

A Vaporizing vehicle is selected from but is not limited to, terpens, propylene glycol and glycerin. The vaporizing vehicle does not contain ethanol or methanol.

Ratio of Proportion of the Ingredients

The pharmaceutical composition of the present invention, comprises of active pharmaceutical agent (API) in the form of particles having an average particle diameter in the range of 0.01 mm to 0.1 mm and having a concentration of about 5% (w/w) to about 45% (w/w), preferably in a concentration of from 7% (w/w) to 35% (w/w), based on the total weight of the pharmaceutical composition.

The ratio of composition of the medicated pill/capsule is illustrated as follows, but is not limited to the indicated examples.

### Example 1

The composition includes API in the proportion of 10-40% and inorganic filler in the proportion of 60-90%.

### Example 2

The composition includes API in the proportion of 10-40% and inorganic filler in the proportion of 10-40% and excipient in the proportion of 20-80%.

### Example 3

The composition includes API in the proportion of 10-40% and excipient in the proportion of 60-90%.

### Example 4

The composition includes API in the proportion of 10-40% and herb fibres in the proportion of 60-90%.

A typical medicated pill/capsule of the present invention comprises of the following ingredients. Their proportion by weight is illustrated in Table 1.

The ingredients include:
Active pharmaceutical ingredient (which should be lipophilic in nature)
Terpenes e.g Limonene, Myrecene, Pinene, Linalool, Terpinolene, Terpineol
Herbs
Fruits and Vegetable extract
Fish oil
Seaweed
Wheatgrass
Preservative
Caffeine
Thickening agent e.g Beeswax

TABLE 1
Formula of Burn Pill per capsule

| Ingredient | Purpose | Amount | |
|---|---|---|---|
| API | Therapeutic activity | Therapeutic dose | |
| Herbs | | 1-2% | w/w |
| Fruits and | | 1-2% | w/w |
| vegetable | | | |
| extract | | | |
| Fish oil | | 500 | mg |
| Seaweed | | 250 | mg |
| Wheatgrass | | 250 | mg |
| Methyl paraben | Preservative | 0.033% | w/w |
| Propyl paraben | Preservative | 0.017% | w/w |

| | | |
|---|---|---|
| Bees Wax | Thickening agent | To desired viscosity |
| Terpenes | Vaporizing agent | This will depend on the size of the |
| | and Vehicle | capsule and density of the other |
| | | excipients. |

For the formulation, the above ingredients in their respective proportions, in each of the above examples are blended with a high shear mixer. A homogenous formulation is obtained after a mixing time between 5 and 30 minutes. The particle size of the formulation is adhered between 0.01 mm and 0.1 mm. The said formulation is then transferred in a holding hopper where the formulation undergoes various procedures for achieving the particular pill design and shape.

The medicated pill when heated, substantially to the boiling or sublimation point of the vaporizing vehicle, usually between the temperature range of 180° to 300° C., the drug volatilizes and when the temperature reaches above 250° C., the medicated pill burns, and therefore, the drug can be inhaled by the user through inhalation by smoking.

The liquid formulation to be filled in the burn pill needs to be a water immiscible/volatile liquid is to be prepared separately. The things to bear in mind while en preparing the fill content are as follows:

1. The Liquid must be viscous enough to allow precise filling of the appropriate volume into the capsule gel. The viscosity should not be more than 25 Pa/s. The addition of thickening agent is critical to the formulation as it influences the stability and viscosity of the fill content. The fill must be viscous enough so that there is uniformity in filling of the capsules and less weight variation. Thickening agents also stabilize the formulation and prevent separation of the content.
2. Chemical and physical interactions could occur between the fill and the shell. An example is crosslinking of gelatin associated with aldehydes that result in solubility problems in the shell. As cross linking occurs in gelatin shells as they get older and when exposed to physical or chemical stress, efforts towards minimizing cross linking, are to be initiated. These are effectuated through the following ways:
i) Addition of succinic acid with two carboxylic acid groups, acts as a moisture barrier in the preparation of the soft gelatin shell with one group masking the amino groups on the amino acids along the gelatin chain while the other providing steric prevention of access of any cross linking agents.
ii) The permissible concentration of succinic acid need not be more than 0.07% of the capsule weight.
iii) Furthermore, as succinic acid is not an excipient for the fill content, it is to be added to the capsule gel only in a way to avoid excipients with aldehyde content.
iv) Addition of anti-oxidants to the formulation of the fill content helps prevent the formation of aldehydes later on. A common anti-oxidant is Ascorbic acid that contains 0.01-0.1% of capsule weight.
v) It is highly recommended to use excipients that also have a free amino group in the soft gelatin shell formulation. An example is glycine which has a free amino group that can compete for aldehyde with gelatin thus limiting cross linking with gelatin.
   3. The water content of the fill content cannot exceed 5% though the formula below doesn't include water or any hydrophilic substance so the water content will be below 5%.
   4. Also, low molecular weight water soluble and volatile organic compounds such as alcohols, ketones, acids, amines, and esters are not suitable for soft gel capsules. Soft gelatin capsules are best suitable for water immiscible/volatile, water immiscible/non-volatile and water miscible/non-volatile liquid fill content. Water miscible compounds that are volatile and have low molecular weight have the tendency to migrate out of the into the gelatin shell and reduce the solubility of the shell. For this reason, these kind of solvents with all three properties are not used for soft gel capsules.

The burn pill is formulated as soft gelatin capsules. These capsules contains a solution of the drug as well as excipients in a suitable solvent which upon ignition, melts away the gelatin, thereby releasing the fill content which is inhaled. The gel capsule is hermetically sealed with the liquid formulation in one process.

This dosage form of the present invention is beneficial for delivering the liquid formulation into the cigarette conveniently and helps to maintain the chemical stability of the active ingredient. Further, these gelatin capsules act as perfect dosage form for administering oils and drugs of low melting point which are related to the Burn Pill of the present invention.

The instant invention is shown and described herein in what is considered to be the most practical and preferred embodiments. It is recognized, however, that departures may be made therefrom which are within the scope of the invention, and that obvious modifications will occur to one skilled in the art upon reading this disclosure.

What is claimed is:

1. A medicated pill having a composition comprising of the active pharmaceutical agent (API) in a concentration in the range of 5% to 40% by weight and excipients in a concentration in the range of 20% to 80% by weight, based on the total weight of the pharmaceutical composition.

2. The medicated pill having a composition of claim 1, wherein the particle diameter of the ingredients of the composition is in the range of 0.01 mm to 0.1 mm.

3. The medicated pill having a composition of claim 1, wherein the active pharmaceutical agent is selected from the group consisting of antibiotics, antipyretics, vitamins and herbs.

4. The medicated pill having a composition of claim 3, wherein the antibiotics is selected from the group consisting of β-lactams, tetracyclines, macrolides, aminoglycosides, anti-metabolites, nucleic acid analogs, antifungals and antivirals.

5. The medicated pill having a composition of claim 3, wherein the antipyretics is chosen from the group consisting of non-steroidal anti-inflammatory drugs.

6. The medicated pill having a composition of claim 1, wherein the excipient selected is organic.

7. The medicated pill having a composition of claim 1, wherein the excipient selected is inorganic.

8. The medicated pill having a composition of claim 6, wherein the organic excipient is selected from the group consisting of cellulose and its derivatives, starch and its derivatives, waxes and herbs.

9. The medicated pill having a composition of claim 7, wherein the inorganic excipient is selected from the group comprising of calcium and sodium salts.

10. The medicated pill having a composition of claim 7, wherein the inorganic excipient is magnesium stearate.

11. The medicated pill having a composition of claim 7, wherein the inorganic excipient is talc.

12. The medicated pill having a composition of claim 7, wherein the inorganic excipient is titanium dioxide.

13. The medicated pill of claim 1, wherein the shape of the pill is cylindrical having a diameter ranging between 3 mm and 8 mm and the length ranging between 5 and 15 mm.

14. The medicated pill of claim 1, wherein the pill is disc shaped having a diameter ranging between 8 and 15 mm and the thickness ranging between 3 and 10 mm.

15. The smoking cylinder for inhaling the vapours of the said medicated pill, having a length ranging between 50 and 250 mm and an inner diameter ranging from 5 and 20 mm.

16. The smoking cylinder of claim 15, wherein an orifice is provided internally in the said smoking cylinder.

17. The smoking cylinder of claim 15, wherein, the end of smoking cylinder is provided with cotton.

18. The smoking cylinder of claim 17, wherein the said cotton end of the said cylinder is blended with a mixture of inorganic salts and polymers.

19. The smoking cylinder of claim 15, wherein the material used for the said cylinder is metal.

20. The smoking cylinder of claim 15, wherein the material used for the said cylinder is glass.

21. The smoking cylinder of claim 15, wherein the material used for the said cylinder is paper made of hemp.
